(19) United States
(12) Patent Application Publication
(10) Pub. No.: US 2005/0287609 A1
(43) Pub. Date: Dec. 29, 2005

(54) Methods and compositions for detection of equine tapeworm infections

(76) Inventors: Stephen A. Kania, Powell, TN (US); Craig R. Reinemeyer, Knoxville, TN (US)

Correspondence Address: KING & SCHICKLI, PLLC, 247 NORTH BROADWAY, LEXINGTON, KY, 40507, US

(21) Appl. No.: 10/770,772

(22) Filed: Feb. 3, 2004

(51) Int. Cl. G01N 33/53; G01N 33/569

(52) U.S. Cl. 435/7.22

(57) ABSTRACT

Methods for detection of a tapeworm in an animal are provided, comprising contacting a biological sample from the animal with at least one antibody capable of binding to an antigen derived from Anoplocephala perfoliata, allowing an antigen-antibody complex to form, separating the biological sample and the antigen-antibody complex, and detecting the antigen-antibody complex. The antigen may be derived by incubating an A. perfoliata organism in a buffer solution, followed by isolating a preparation containing molecules having a molecular weight of at least about 100 kD from the incubated solution and separating at least one antigen having a molecular weight of from about 12 to about 13 kD from the preparation. Compositions for accomplishing the methods, and kits comprising the compositions are provided.

### TECHNICAL FIELD

The present invention relates to methods and compositions for detection and diagnosis of equine tapeworm infections. More specifically, the present invention relates to immunoassays for detection of Anoplocephala perfoliata in fecal samples. The invention further relates to antigens and antibodies for use in the immunoassays.

### BACKGROUND OF THE INVENTION

Tapeworm infections are a common intestinal parasite of horses, with a prevalence of as high as 60% in certain geographical areas. The most common species of equine tapeworm is Anoplocephala perfoliata, also known as the cecal tapeworm. Other species of equine tapeworm include Anoplocephala magna, which locates in the posterior small intestine, and Paranoplocephala mamillana, typically found in the anterior small intestine and occasionally the stomach.

Detrimental effects of tapeworms, particularly A. perfoliata, include intestinal blockage, ulceration, inflammation, and formation of a diphtheritic membrane at the site of parasite attachment. Other pathologic effects have been reported, particularly in younger animals, including intestinal perforation, intussusception (prolapse) of the terminal ileum and cecum, and hypertrophy/hyperplasia (thickening) of the ileal walls. In severe cases, abdominal surgery is required to alleviate these effects.

Conventional drugs used to control equine parasites are substantially ineffective against equine tapeworms, although new products exist and additional drugs are in various stages of development. Indiscriminate use of such drugs is not recommended. For example, pyrantel pamoate, which is marketed for removal of nematodes, has some effect on A. perfoliata. However, at the therapeutic dose rate (6.6 mg base/kg), the drug is less effective than at the double dose rate (13.2 mg base/kg). At this higher dose, safety in breeding animals has not been determined, and accordingly increasing the drug amounts beyond the approved therapeutic dose is not recommended, particularly in pregnant mares. It is therefore important to be able to accurately diagnose the presence of a tapeworm infection such as by A. perfoliata to be able to target drug use only to animals specifically in need of treatment. Standard techniques for detection of other parasites involve detection of parasite eggs in feces, or centrifugation/flotation techniques for identifying eggs in feces. Such techniques are not reliable for tapeworms, being unreliable, expensive, and labor-intensive.

A blood test for detection of A. perfoliata antibody has been developed and correlated with tapeworm infection intensity (Proudman, C. J. and A. J. Trees, 1996, Use of excretory/secretory antigens for the serodiagnosis of Anoplocephala perfoliata cestodosis, Veterinary Parasitology 61: 239-247; Proudman, C. J. and A. J. Trees, 1996, Correlation of antigen specific IgG and IgG(T) responses with Anoplocephala perfoliata infection intensity in the horse, Parasite Immunology: 499-506; both incorporated herein in their entirety by reference). This test is effective for its intended use. However, such tests measure serological concentrations of antibody to A. perfoliata antigens, and therefore determine only that the animal has been exposed to the tapeworm, rather than detecting actual ongoing infections.

Accordingly, there remains a need in the art for a reliable test for detecting the presence of an ongoing tapeworm infection in an animal, rather than simply a past exposure to the organism, to allow for more efficient treatment with minimal drug use. The present invention satisfies this need by providing a sensitive, accurate method for detecting the presence of tapeworm antigens shed into feces. Still further, the present invention provides compositions suitable for accomplishing the method, and a kit for accomplishing the method which provides a convenient, animal-side test for A. perfoliata infection.

### SUMMARY OF THE INVENTION

In one aspect of the present invention, a method is provided for detecting or diagnosing the presence of an Anoplocephala perfoliata organism in an animal, comprising contacting a biological sample from the animal with at least one antibody capable of binding to an antigen derived from A. perfoliata, allowing an antigen-antibody complex to form, separating the biological sample and the antigen-antibody complex, and detecting the antigen-antibody complex. The antigen is derived by the steps of incubating an A. perfoliata organism in a buffer solution, isolating a preparation containing molecules having a molecular weight of at least about 100 kD from the incubated solution, and separating at least one antigen having a molecular weight of from about 12 to about 13 kD from the preparation.

Typically, the biological sample is a fecal sample. The antigen may be secreted or excreted by an A. perfoliata organism in the animal's intestinal tract, and is then excreted with the feces. A preferred method for detecting the antigen-antibody complex is an immunoassay. It will be appreciated that a number of immunoassays known in the art may be suitable for detecting the antigen, for example an enzyme-linked immunosorbent assay, a radioimmunoassay, complement fixation, indirect hemagglutination, latex agglutination, a rapid flow-through assay, a lateral flow assay, a fluoroimmunoassay, a chemoluminescent immunoassay, an enzyme immunoassay, an antigen capture enzyme-linked immunosorbent assay, an enzyme multiplied immunoassay, a competitive enzyme-linked immunosorbent assay, and the like. In a presently preferred embodiment of this invention, the antigen-antibody complex is detected using an antigen capture enzyme-linked immunosorbent assay.

In another aspect of the present invention, a composition is provided for use in detecting or diagnosing the presence of an A. perfoliata organism in an animal, comprising at least one antibody capable of binding to an antigen derived from A. perfoliata, the antigen being contained in a biological sample such as fecal matter. The antigen may be derived as described above.

In yet another aspect of this invention, a kit is provided for detecting or diagnosing the presence of an A. perfoliata infection in an animal, comprising a first antibody and a second antibody each capable of binding to at least one A. perfoliata antigen contained in a biological sample. The antigen is as described above. Typically, the biological sample will be a fecal sample.

The first antibody may be immobilized on a solid support. Typically, the second antibody will include a marker. The marker may be selected from the group consisting of biotin, a peroxidase, fluorescein, avidin, streptavidin, a phosphatase, and the like. A detecting substance for identifying the presence of the marker may be provided, along with a substrate for identifying the presence of the detecting substance bound to the marker. The substrate may be a chromogenic substrate, a chemiluminescent substrate, or the like.

In still yet another aspect of this invention, a method is provided for detecting or diagnosing the presence of an A. perfoliata infection in an animal, comprising dispersing a fecal sample obtained from the animal in a diluent, contacting a supernatant of the dispersed fecal sample with at least one antibody capable of binding to the antigen, allowing an antigen-antibody complex to form, separating the supernatant and the antigen-antibody complex, and detecting the antigen-antibody complex. The antigen may be obtained as described above.

The dispersion of the fecal sample may comprise dispersing the fecal sample in a first diluent, obtaining a supernatant of the dispersed fecal sample, and diluting the supernatant in a second diluent containing a blocker for inhibiting non-specific binding of the first antibody. The blocker may be a proteinaceous substance such as fetal bovine serum.

As noted above, the antigen-antibody complex may be detected by any suitable immunoassay, such as for example an enzyme-linked immunosorbent assay, a radioimmunoassay, complement fixation, indirect hemagglutination, latex agglutination, a rapid flow-through assay, a lateral flow assay, a fluoroimmunoassay, a chemoluminescent immunoassay, an enzyme immunoassay, an antigen capture enzyme-linked immunosorbent assay, an enzyme multiplied immunoassay, a competitive enzyme-linked immunosorbent assay, and the like. In one typical embodiment, the antigen-antibody complex is detected using an antigen capture enzyme-linked immunosorbent assay. The antibody may be immobilized on a solid substrate.

In still yet another aspect of the present invention, there is provided a kit for detecting or diagnosing the presence of an A. perfoliata antigen in a fecal sample, comprising a first antibody capable of binding to the antigen, a second antibody capable of binding to the antigen wherein the second antibody includes a marker, at least one diluent for diluting the fecal sample, a detecting substance for identifying the presence of the marker, and a substrate for identifying the presence of the detecting substance. Preparation of the antigen has been described supra.

The first antibody may be immobilized on a suitable solid support. Numerous suitable solid supports are known in the art, for example a multi-well culture dish or test plate such as a 96-well microtiter plate. The second antibody marker may be any suitable antibody marker molecule, such as for example biotin, a peroxidase, fluorescein, avidin, streptavidin, a phosphatase, or the like. The detecting substance will be selected in accordance with the marker chosen, and any suitable detecting substance may be selected, such as an avidin-peroxidase conjugate, an avidin-phosphatase conjugate, or the like. The substrate may be a chromogenic substrate to generate a detectable and/or quantifiable color response signifying the presence of the antigen-antibody complex. The substrate may also be a chemiluminescent substrate for generating a detectable light response, selected in accordance with the detecting substance of choice.

A standard antigen preparation for quantitating the amount of A. perfoliata antigen in the fecal sample may also be included, being a predetermined range of antigen concentrations. The kit may further include a first diluent for dispersing the fecal sample and a second diluent containing a blocker for inhibiting non-specific binding of the first antibody. The second diluent blocker is typically a proteinaceous substance such as fetal bovine serum.

As should be appreciated, the embodiment shown and described herein is an illustration of one of the modes best suited to carry out the invention. It will be realized that the invention is capable of other different embodiments and its several details are capable of modification in various, obvious aspects all without departing from the invention. Accordingly, the drawings and descriptions will be regarded as illustrative in nature and not as restrictive.

### BRIEF DESCRIPTION OF THE DRAWINGS

The accompanying drawings incorporated in and forming a part of the specification, illustrate several aspects of the present invention and together with the description serve to explain the principles of the invention. In the drawings:

FIG. 1 shows a typical antigen capture ELISA standard curve for A. perfoliata antigen; and

FIG. 2 shows detection of A. perfoliata antigen using an antigen capture ELISA in fecal samples obtained from horses with confirmed tapeworm infections and from horses known to be tapeworm negative.

Reference will now be made in detail to the present preferred embodiment of the invention, an example of which is illustrated in the accompanying drawings.

### DETAILED DESCRIPTION OF THE INVENTION

In accordance with the identified need in the art, a method for detecting the presence of an A. perfoliata antigen in a biological sample is provided. The method allows detection or diagnosis of the presence of A. perfoliata in an animal, and comprises contacting a biological sample from the animal with at least one antibody capable of binding to an antigen derived from A. perfoliata, allowing an antigen-antibody complex to form, separating the biological sample and the antigen-antibody complex, and detecting the antigen-antibody complex. The method described herein is suited to detection of A. perfoliata antigens in fecal samples, and thereby advantageously provides an indicator of an ongoing tapeworm infection rather than merely an indicator of past exposure as is the case for serological immunoassays measuring amounts of A. perfoliata antibody in serum.

The antibody is raised against an antigen derived by the steps of incubating an A. perfoliata organism in a buffer solution, isolating a preparation containing molecules having a molecular weight of at least about 100 kD from the incubated solution, and separating at least one antigen having a molecular weight of from about 12 to about 13 kD from the preparation. The embodiments described herein comprise polyclonal antibodies, produced by sensitizing an animal to the antigen as described above. However, it will be appreciated that the use of monoclonal antibodies, produced by methods well known in the art, is also encompassed by the present invention. It will also be appreciated that rather than using native A. perfoliata antigen, the entirety of the antigen, or particularly immunogenic portions thereof, may be recombinantly derived as a synthetic peptide or peptides by methods well known in the art, and subsequently used to sensitize an animal to obtain a polyclonal antibody, or to produce a monoclonal antibody.

A typical method for detecting the antigen-antibody complex is an immunoassay. It will be appreciated by skilled artisans in this field that a number of immunoassays known in the art may be adapted for use in detecting the antigen, such as for example an enzyme-linked immunosorbent assay, a radioimmunoassay, complement fixation, indirect hemagglutination, latex agglutination, a rapid flow-through assay, a lateral flow assay, a fluoroimmunoassay, a chemoluminescent immunoassay, an enzyme immunoassay, an antigen capture enzyme-linked immunosorbent assay, an enzyme multiplied immunoassay, a competitive enzyme-linked immunosorbent assay, and the like, as well as combinations thereof. In one typical embodiment of this invention, the antigen-antibody complex is detected using an antigen capture enzyme-linked immunosorbent assay.

Still further, the present invention provides a kit including compositions for detecting or diagnosing the presence of an A. perfoliata antigen in a biological sample such as a fecal sample. The kit may include a first or capture antibody capable of binding to the antigen, a second or detecting antibody capable of binding to the antigen, wherein the second antibody includes a marker, a detecting substance for identifying the presence of the marker, a substrate for identifying the presence of the detecting substance, and at least one diluent for diluting the fecal sample.

The first antibody may be immobilized on a suitable solid support prior to contact with the diluted fecal sample. Numerous suitable solid supports are known in the art, for example a multi-well culture or test plate such as a 96-well microtiter plate. The second antibody marker may be any suitable antibody marker molecule, such as for example biotin, a peroxidase, fluorescein, avidin, streptavidin, a phosphatase, or the like. The detecting molecule will be selected in accordance with the selected marker, such as for example an avidin-peroxidase conjugate or an avidin-phosphatase conjugate for use with a biotinylated antibody. The substrate may be a chromogenic substrate to generate a detectable and/or quantifiable color response signifying the presence of the antigen-antibody complex. Alternatively, the substrate may be a chemiluminescent substrate generating a detectable/quantifiable light reaction which may be detected by any known art means, such as X-ray film or a luminometer.

A standard antigen preparation for quantitating the amount of A. perfoliata antigen in the fecal sample may also be included, comprising a predetermined range of antigen concentrations. The diluent may comprise a first diluent for dispersing the fecal sample and a second diluent containing a blocker for inhibiting non-specific binding of the first antibody. The second diluent blocker is typically a proteinaceous substance such as fetal bovine serum.

### EXAMPLE 1

A. perfoliata crude antigen was prepared for use in immunoassays as described in greater detail below. Briefly, A. perfoliata were collected from the intestines of infected horses and maintained in a buffered saline solution. After 5 hours incubation time, the cultures were centrifuged and the supernatant collected.

To confirm the presence of antigenic/immunogenic components, the supernatant was subjected to molecular sieve chromatography in Bio-gel P10. Separated supernatant fractions containing A. perfoliata antigen (500 μg/ml) were then mixed with SDS-PAGE sample buffer (Sigma, St. Louis, Mo.), boiled for 5 minutes, and electrophoresed on an 18% SDS-PAGE gel (Bio-Rad Laboratories, Hercules, Calif.) in Tris-HCl buffer at 120 volts. The electrophoresed fractions were then transferred to nitrocellulose (60 volts for 2 hours), washed with PBS-tween 20 (0.05%), and probed with sera obtained from a known tapeworm-positive horse. The presence of antibody to A. perfoliata antigen was then detected using anti-equine IgG(T) labeled with phosphatase, and a chromogenic substrate. Surprisingly, it was found that the most immunogenic proteins eluted as a molecule or molecules having a molecular weight of greater than 100 kD.

### EXAMPLE 2

A. perfoliata supernatant prepared as described in Example 1 was centrifuged, passed through a 0.22 μm filter, and then passed through a 100 kD NMWCO filter (Centricon Plus-20) to separate the most immunogenic molecules, i.e. those having a molecular weight of at least 100 kD. The antigens retained on the filter were then further purified by SDS-PAGE. The retained antigens were mixed with SDS-PAGE buffer and boiled for 6 minutes. Next, the antigen preparations were applied to 18% SDS-PAGE gels and separated in accordance with the manufacturer's directions. Reference gel strips were stained, and gel bands corresponding to proteins having a molecular weight of from about 12 to about 13 kD were removed.

Each gel band was then placed into the gel holder of a Centrilutor micro eluter (Amicon; Millipore, Billerica, Mass.). The antigens were eluted into Millipore (Billerica, Mass.) ultrafree Biomax 10 k NMWC membranes using degassed 0.025 M tris, 0.192 M glycine buffer at pH 8.4 and 200 volts constant voltage. The retained samples containing the desired antigen were then concentrated and exchanged into phosphate-buffered saline.

### EXAMPLE 3

Antigen prepared as described in Example 2 was emulsified in incomplete Freund's adjuvant and injected into a New Zealand white rabbit (20 μg antigen per injection, total of three injections spaced three weeks apart). The rabbit was bled, and rabbit IgG was enriched using a protein A kit (Pierce, Rockford, Ill.) in accordance with the manufacturer's directions. The enriched IgG was exchanged into PBS (0.1 M, pH 7.5, GIBCO) using a 10 K NMW cutoff membrane (Millipore, Billerica, Mass.), and concentrated (2 mg/ml). Labeled antibody was prepared by coupling to EZ link sulfo-NHS-LC biotin (Pierce, Rockford, Ill.) in accordance with the manufacturer's instructions. Free biotin was separated using a centrifugal filter (Millipore, Billerica, Mass.). Labeled antibody was exchanged into PBS and stored at −80° C.

### EXAMPLE 4

An antigen capture ELISA was developed using the antibody and antigen prepared as described in Examples 1-3. Briefly, unlabeled antibody to A. perfoliata antigen (prepared as described in Example 3) was diluted in PBS and coated overnight at 4° C. on Immulon 2 96-well microtiter plates (Dynatech Laboratories, Chantilly, Va.). Each well was washed three times with PBS-tween 20 (0.05%) buffer. Antigen in dilution buffer was added at varying concentrations increasing incrementally from 10 ng/ml to 10,000 ng/ml (100 μL/well) and incubated for 1 hour at 37° C. Each well was then washed, and biotin-labeled antibody diluted in PBS-tween 20 buffer was added (100 μl/well) and incubated for 1 hour at 37° C. Each well was washed, and peroxidase-conjugated avidin was added. After incubation at 37° C. for 1 hour, each well was washed to remove unbound avidin, and peroxidase substrate (KPL, Inc., Gaithersburg, Md.) was added at room temperature (100 μl/well). The optical density in each well was determined at a 405 nm wavelength. The optimal concentrations for the first (capturing) antibody and the second (labeled or detecting) antibody were determined by titration.

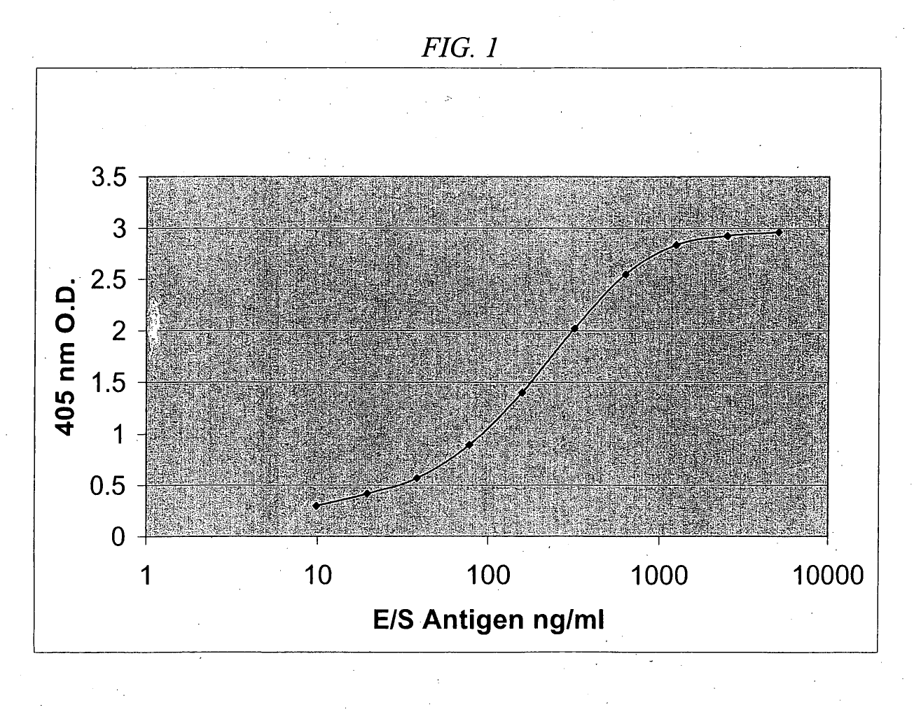

The results of a representative antigen standard curve are presented in FIG. 1. At the concentrations of antigen tested, plotting the results of the antigen capture ELISA generated a standard sigmoid curve.

### EXAMPLE 5

For detection of A. perfoliata antigen in feces, fecal samples were collected from horses having A. perfoliata eggs detected in the manure. Negative fecal samples were collected from horses known to be free of A. perfoliata infection. Two grams of feces were mixed with 8 ml of PBS by shaking, and centrifuged at 2,200×g for 10 minutes. The supernatant was collected, and microcentrifuged at 21,000×g for 5 minutes. This microcentrifuged supernatant was retained for further analysis.

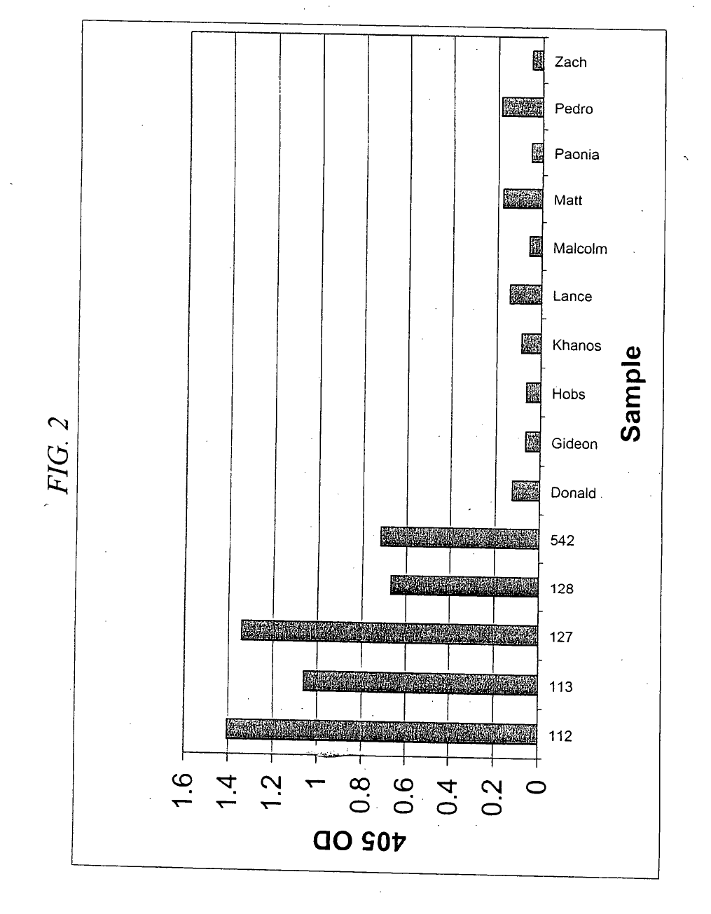

The microcentrifuged supernatant was diluted 1:50 in either 0.5% or 0.25% fetal bovine serum in PBS-0.05% tween 20 and analyzed using the antigen capture ELISA described in Example 4. Fetal bovine serum was included to block non-specific binding by the unlabeled antibody. Results of analyses using 0.5% FBS are shown in FIG. 2. Significant concentrations of A. perfoliata antigen were detected only in the horses known to be positive for tapeworm infection (nos. 112, 113, 127, 128, and 542). Samples from horses known to be free of tapeworm did not contain significant concentrations of the antigen in their fecal samples.

Accordingly, a method has been described for the detection or diagnosis of A. perfoliata infections in animals such as horses, which advantageously allows the detection of an ongoing infection rather than merely the detection of prior exposure to the organism. The present invention provides compositions for carrying out the method, and a diagnostic kit which may be adapted to animal-side testing, thereby increasing the convenience thereof. By use of the present invention it is possible to accurately and rapidly screen, for example, a herd of horses for A. perfoliata infection and to treat only the actually infected animals.

The foregoing description of a preferred embodiment of the invention has been presented for purposes of illustration and description. It is not intended to be exhaustive or to limit the invention to the precise form disclosed. Obvious modifications or variations are possible in light of the above teachings. For example, it is well known in the art to produce antigens as synthetic peptides or recombinant proteins, comprising all or portions of immunogenic molecules, and to use such synthetic peptides to produce polyclonal or monoclonal antibodies for use in immunoassays such as have been described herein.

The embodiment was chosen and described to provide the best illustration of the principles of the invention and its practical application to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally and equitably entitled.

1. A method for detecting or diagnosing the presence of an Anoplocephala perfoliata organism in an animal, comprising:
contacting a biological sample from the animal with at least one antibody capable of binding to an antigen derived from Anoplocephala perfoliata;
allowing an antigen-antibody complex to form;
separating the biological sample and the antigen-antibody complex; and
detecting the antigen-antibody complex;
the method being characterized by deriving the antigen by the steps of:
incubating an Anoplocephala perfoliata organism in a buffer solution;
isolating a preparation containing molecules having a molecular weight of at least about 100 kD from the incubated solution; and
separating at least one antigen having a molecular weight of from about 12 to about 13 kD from the preparation.

2. The method of claim 1, wherein the biological sample is a fecal sample.

3. The method of claim 1, wherein the antigen-antibody complex is detected by an immunoassay.

4. The method of claim 3, wherein the immunoassay is selected from the group consisting of an enzyme-linked immunosorbent assay, a radioimmunoassay, complement fixation, indirect hemagglutination, latex agglutination, a rapid flow-through assay, a lateral flow assay, a fluoroimmunoassay, a chemoluminescent immunoassay, an enzyme immunoassay, an enzyme multiplied immunoassay, an antigen capture enzyme-linked immunosorbent assay, a competitive enzyme-linked immunosorbent assay, and any combination thereof.

5. The method of claim 4, wherein the antigen-antibody complex is detected by an antigen capture enzyme-linked immunosorbent assay.

6. A composition for use in detecting or diagnosing the presence of an Anoplocephala perfoliata organism in an animal, comprising:
at least one antibody capable of binding to an antigen derived from Anoplocephala perfoliata, the antigen being contained in a biological sample; and
the composition being characterized by deriving the antigen by the steps of:
incubating an Anoplocephala perfoliata organism in a buffer solution;
isolating a preparation containing molecules having a molecular weight of at least about 100 kD from the incubated solution; and
separating at least one antigen having a molecular weight of from about 12 to about 13 kD from the preparation.

7. The composition of claim 6, wherein the biological sample is a fecal sample.

8. A kit for detecting or diagnosing the presence of an Anoplocephala perfoliata infection in an animal, comprising a first antibody and a second antibody each capable of binding to at least one Anoplocephala perfoliata antigen contained in a biological sample;
the antigen being derived by the steps of:
incubating an Anoplocephala perfoliata organism in a buffer solution;
isolating a preparation containing molecules having a molecular weight of at least about 100 kD from the incubated solution; and
separating at least one antigen having a molecular weight of from about 12 to about 13 kD from the preparation.

9. The kit of claim 8, wherein the biological sample is a fecal sample.

10. The kit of claim 8, wherein the first antibody is immobilized on a solid support.

11. The kit of claim 8, wherein the second antibody includes a marker.

12. The kit of claim 11, wherein the marker is selected from the group consisting of biotin, a peroxidase, fluorescein, avidin, streptavidin, and a phosphatase.

13. The kit of claim 12, further including a detecting substance for identifying the presence of the marker.

14. The kit of claim 13, wherein the detecting substance is selected from the group consisting of an avidin-peroxidase conjugate and an avidin-phosphatase conjugate.

15. The kit of claim 12, further including a substrate for identifying the presence of the detecting substance bound to the marker.

16. The kit of claim 15, wherein the substrate is selected from the group consisting of a chromogenic substrate and a chemiluminescent substrate.

17. A method for detecting or diagnosing the presence of an Anoplocephala perfoliata infection in an animal, comprising:
dispersing a fecal sample obtained from the animal in a diluent;
contacting a supernatant of the dispersed fecal sample with at least one antibody capable of binding to the antigen;
allowing an antigen-antibody complex to form;
separating the supernatant and the antigen-antibody complex; and
detecting the antigen-antibody complex.

18. The method of claim 17, wherein the antigen is obtained by the steps of:
incubating an Anoplocephala perfoliata organism in a buffer solution;
isolating a preparation containing molecules having a molecular weight of at least about 100 kD from the incubated solution; and
separating at least one antigen having a molecular weight of from about 12 to about 13 kD from the preparation.

19. The method of claim 17, wherein dispersion of the fecal sample comprises the steps of:
dispersing the fecal sample in a first diluent;
obtaining a supernatant of the dispersed fecal sample; and
diluting the supernatant in a second diluent containing a blocker for inhibiting non-specific binding of the first antibody.

20. The method of claim 17, wherein the antigen-antibody complex is detected by an immunoassay.

21. The method of claim 20, wherein the immunoassay is selected from the group consisting of an enzyme-linked immunosorbent assay, a radioimmunoassay, complement fixation, indirect hemagglutination, latex agglutination, a rapid flow-through assay, a lateral flow assay, a fluoroimmunoassay, a chemoluminescent immunoassay, an enzyme immunoassay, an enzyme multiplied immunoassay, an antigen capture enzyme-linked immunosorbent assay, a competitive enzyme-linked immunosorbent assay, and any combination thereof.

22. The method of claim 21, wherein the antigen-antibody complex is detected by an antigen capture enzyme-linked immunosorbent assay.

23. The method of claim 17, wherein the antibody is immobilized on a solid support.

24. The method of claim 19, wherein the second diluent blocker is a proteinaceous substance.

25. The method of claim 24, wherein the blocker is fetal bovine serum.

26. A kit for detecting or diagnosing the presence of an Anoplocephala perfoliata antigen in a fecal sample, comprising:
a first antibody capable of binding to the antigen;
a second antibody capable of binding to the antigen, wherein the second antibody includes a marker;
at least one diluent for diluting the fecal sample;
a detecting substance for identifying the presence of the marker; and
a substrate for identifying the presence of the detecting substance.

27. The kit of claim 26, wherein the antigen is derived by the steps of:
incubating an Anoplocephala perfoliata organism in a buffer solution;
isolating a preparation containing molecules having a molecular weight of at least about 100 kD from the incubated solution; and
separating at least one antigen having a molecular weight of from about 12 to about 13 kD from the preparation.

28. The kit of claim 26, wherein the first antibody is immobilized on a solid support.

29. The kit of claim 28, wherein the solid support is a multi-well culture dish.

30. The kit of claim 26, wherein the marker is selected from the group consisting of biotin, a peroxidase, fluorescein, avidin, streptavidin, and a phosphatase.

31. The kit of claim 26, wherein the detecting substance is selected from the group consisting of an avidin-peroxidase conjugate and an avidin-phosphatase conjugate.

32. The kit of claim 26, wherein the substrate is selected from the group consisting of a chromogenic substrate and a chemiluminescent substrate.

33. The kit of claim 26, further including a standard antigen preparation for quantitating an Anoplocephala perfoliata antigen, the standard antigen preparation comprising a predetermined range of antigen concentrations.

34. The kit of claim 26, wherein is included:
a first diluent for dispersing the fecal sample; and
a second diluent containing a blocker for inhibiting non-specific binding of the first antibody.

35. The kit of claim 34, wherein the second diluent blocker is a proteinaceous substance.

36. The kit of claim 35, wherein the blocker is fetal bovine serum.
